(19) United States
(12) Patent Application Publication
(10) Pub. No.: US 2026/0215746 A1
(43) Pub. Date: Jul. 30, 2026

(54) MULTIMODALITY DUAL-ENDED WIRELESS SURGICAL PROBE SYSTEM

(71) Applicant: Nedventive LLC, New York, NY (US)

(72) Inventor: Nicole Esther Daghighian, New York, NY (US)

(21) Appl. No.: 19/578,589

(22) Filed: Mar. 25, 2026

Related U.S. Application Data
(63) Continuation-in-part of application No. 18/668,205, filed on May 19, 2024.
(60) Provisional application No. 63/467,837, filed on May 19, 2023.

(51) Int. Cl. A61B 6/00; A61B 6/42; A61B 6/46

(52) U.S. Cl. CPC A61B 6/4405; A61B 6/4216; A61B 6/425; A61B 6/4258; A61B 6/4411; A61B 6/4417; A61B 6/462; A61B 6/465; A61B 6/467; A61B 6/54; A61B 6/56

(57) ABSTRACT

An ergonomic, wireless multimodality surgical probe system provides intraoperative detection of Gamma radiation, RFID tags, and tissue autofluorescence. The probe integrates an autofluorescence module configured to excite tissue (300-800 nm) and detect metabolic emission signals (400-850 nm). The handheld probe features a central battery compartment that functions as an electromagnetic interference attenuating structure, physically shielding a distal radiation sensor assembly from a proximal wireless antenna. The system integrates a compact control console with a bi-directional graphical user interface. Specialized structural architectures accommodate modular open surgical procedures utilizing bayonet-locking interchangeable segments, and robotic-assisted minimally invasive procedures utilizing a bundled array of continuous, simultaneously deployable sensors for intact insertion through a single surgical trocar.

### CROSS REFERENCE TO RELATED APPLICATIONS

The present non-provisional patent application is Continuation-In-Part application of U.S. Non-Provisional patent application Ser. No. 18/668,205 filed May 19, 2024, which claims the benefit of and priority to U.S. Provisional Patent Application 63/467,837 filed May 19, 2023. The contents of each of the Ser. No. 18/668,205 and 63/467,837 applications are incorporated herein by reference in their entireties.

### FIELD OF THE DISCLOSURE

The present invention relates generally to surgical detection systems and more particularly to a multimodality dual-ended wireless surgical probe system.

### BACKGROUND

Multimodality probes have advanced the precision of intraoperative lesion localization, offering surgeons real-time detection via Tc-99m, I-125 seeds, RFID markers, Magseed®, fluorescence contrast, ultrasound, and white light imaging. However, there exists a need for integration of endogenous autofluorescence capability to distinguish tissue morphology without the use of injected fluorophores. Autofluorescence imaging allows differentiation of normal and pathological tissue based on naturally occurring fluorophores from chemical compounds such as Nicotinamide Adenine Dinucleotide Phosphate (NADH), Flavin Adenine Dinucleotide (FAD), melanin, and lipofuscin. These are examples of compounds that are often present in reduction and oxidation on a cellular level, which results in photon emission at certain wavelengths. When a surgeon excites these photons using light at certain wavelengths, corresponding lenses absorb and display those as images.

Autofluorescence allows surgeons to utilize the proper levels of photon emission from electron excitation and lens absorption to ablate target tissue without injecting fluorescent dyes, radioactive tracers, magnetic seeds, or radiofrequency tags. Injecting radioactivity is often still done in cases where suspect lesions may be too deep to detect through autofluorescence. That is why a multimodality probe allowing surgical staff to run certain functions simultaneously or in tandem can save hospitals vital time and money.

Removal of suspect lesions in early-stage cancers is often assisted by wire localization or radioactive seed localization. Probes that detect lesions in the body for cancer include gamma probes, magnetic seed localization probes, and Radio Frequency Identification (RFID) probes. Each of these devices consumes space in the operating room.

For a lesion within a breast to be removed during a surgical procedure such as lumpectomy or biopsy, the lesion's location must be identified. Surgeons have various techniques to locate breast lesions that are both two-dimensional and three-dimensional. Three-dimensional procedures include radioactive seed localization and RFID. Both involve inserting a small consumable “tag” into the breast lesion with the aid of mammography or ultrasound prior to surgery, and during the surgery with the aid of a handheld detector, the surgeon will detect these tags and through them the lesion during surgery.

Tracers are playing an increasingly important role in enhancing surgical precision. Tracers come in various forms, each offering unique advantages depending on the surgical scenario. Surgical procedures have become increasingly precise thanks in part to the use of specialized tracers. Tracers highlight specific tissues or structures, guiding surgeons and enhancing the accuracy of minimally invasive techniques.

Below is a summary of commonly used tracers and their detection systems:
Technetium-99 (99mTc): This radioactive tracer plays a vital role in nuclear medicine scans performed before and during surgery. 99mTc attaches to molecules that target specific tissues. 99mTc can be used in cancer surgery to identify tumors or sentinel lymph nodes, the first lymph nodes to receive drainage from a tumor.

Surgeons use a gamma probe to detect the gamma rays emitted by 99mTc, guiding the surgeon to detect the sentinel nodes in the patient. In breast cancer cases, these sentinel nodes are in the patient's axilla. The sentinel nodes are sent to pathology and help with mapping and staging of the patient's cancer.

Iodine-125 (I-125): Another radioactive tracer, Iodine-125, is often injected into the patient's tumors, and assists in identifying lesions.

Radio Frequency Identification (RFID) tags: Transitioning to the realm of intraoperative guidance, RFID tags offer real-time tracking during surgery. These tiny tags, often no larger than a grain of rice, can be attached to tissues. A handheld scanner emits radio waves that the tags respond to, revealing their precise location. This technology is particularly beneficial in minimally invasive procedures, where surgeons have limited visual access to the operative field.

Fluorescence imaging has been increasingly utilized in lesion detection during surgery, particularly in oncological and neurosurgical procedures. Primary uses of fluorescence in lesion detection during surgery include tumor visualization in which fluorescent dyes or probes may be administered to the patient prior to surgery. These dyes specifically target cancer cells or tumor-associated biomarkers. When illuminated with an appropriate light source, such as a near-infrared laser, the cancerous tissue fluoresces or illuminates, allowing surgeons to more rapidly visualize and accurately delineate tumor margins.

Fluorescence imaging is also used in sentinel lymph node mapping. During procedures involving the removal of lymph nodes, such as in breast cancer or melanoma surgeries, fluorescent dyes are injected near the tumor site. These dyes migrate to the sentinel lymph nodes, which are the first nodes to receive drainage from the tumor. By using fluorescence imaging, surgeons can identify and selectively remove these sentinel lymph nodes, reducing the risk of unnecessary lymph node dissection.

Fluorescence imaging distinguishes healthy tissue from diseased tissue during surgery. By targeting specific biomarkers associated with disease, fluorescent probes can highlight areas of abnormal tissue, allowing surgeons to precisely remove diseased tissue while preserving healthy surrounding tissue.

Fluorescence imaging provides real-time assessment of tissue perfusion. In surgeries where tissue perfusion is critical, such as vascular or reconstructive surgeries, fluorescence imaging can provide real-time assessment of blood flow. Indocyanine green (ICG) is a fluorescent dye commonly used for this purpose. By injecting ICG into the bloodstream, surgeons can monitor tissue perfusion by visualizing the fluorescence intensity in the target tissue, helping to guide surgical decisions and optimize outcomes.

In neurosurgery, fluorescence-guided imaging can aid in the visualization and resection of brain tumors, particularly gliomas. Tumor cells often infiltrate surrounding healthy brain tissue, making it challenging to distinguish tumor margins. Fluorescent dyes targeting tumor-specific biomarkers can help surgeons identify tumor boundaries more accurately, reducing the risk of leaving behind residual tumor tissue.

Overall, fluorescence imaging offers valuable advantages in lesion detection during surgery, including improved visualization of tumor margins, enhanced precision in tissue resection, and real-time assessment of tissue perfusion. As technology continues to advance, fluorescence-guided surgery is expected to play an increasingly important role in improving surgical outcomes across various medical specialties.

Multiple modalities are often needed during different phases of surgery. For example, the surgeon may require a handheld RFID detector to locate the RFID tag that was left inside the tumor during mammography or ultrasonography. Also, for the localization of the sentinel lymph node objected by a radiotracer such as 99mTcm sulfur colloid, the surgeon utilizes another hand-held detector of gamma ray.

In the context of ear, nose and throat (ENT) surgeries, autofluorescence is a form of detecting parathyroid adenoma from the thyroid without injecting any fluorescent tracers. Parathyroid tissue has fluorophores, a compound that emits light when examined through a violet/blue light source (~785 nm excitation, ~820-830 nm emission). The target compound that releases fluorophores in that tissue in the parathyroid are within the Near Infrared range, while some may be better seen with an injectable tracer. Autofluorescence can be used to visualize potential margins of cancer tumors in patients. Since the compounds that release fluorophores when excited by certain wavelengths, a clinician may find it beneficial to view all simultaneously. Being able to resect the most amount of margins by being able to see emissions from flavins, NADH, melanin and lignin emission profiles could lead to better margin resection.

When surgeons are removing a tumor lesion with the use of an I-125 radioactive seed, they can often use a gamma detection probe, the same probe they already intend to use for sentinel node mapping later in the surgical procedure. However, if the surgeon chooses to use RFID for their procedure, he or she would need another probe in the operating room. This would mean another apparatus to have in the sterile field and insert into the patient.

Existing technologies include detector probes for at least: 1) 99mTcm isotope that emit 140 keV gamma rays for sentinel node biopsy, 2) I-125 isotope in the form of seed for tumor localization, 3) radio wave antenna for localization of the RFID tags for tumor localization, 4) mini-camera or detector for fluorescent detection of immunofluorescent dye for various tissue characterization such as blood perfusion, 5) Ultrasound transducer for ultrasound imaging, 6) visible light detector for imaging the field of view, 7) magnetic field detectors for localizing Magseed®.

Multimodality probes have advanced the precision of intraoperative lesion localization, offering surgeons real-time detection via Tc-99m, I-125 seeds, RFID markers, Magseed®, fluorescence contrast, ultrasound, and white light imaging. These modalities can improve quality of life by increasing the surgeon's capability to detect and resect suspect lesions more accurately. While imaging modalities have expanded, the sizes of Operating Rooms (OR) have remained the same. Additionally, surgeons needing to increase their already high cognitive load to widen their field of view to switch devices leads to longer case times. A device which has the modalities integrated into one system can leave a smaller footprint in the OR, lower the cognitive load for surgeons and OR staff, and decrease the capital equipment costs for hospitals long term.

A device which has multiple (e.g., all) modalities integrated into one system (one unified control and display unit, one connecting cable, and a multi-detector hand-held piece) can leave a smaller footprint in the OR, lower the cognitive load for surgeons and OR staff, reduce the number of items to be re-sterilized, and decrease the capital equipment costs for hospitals long term. This unified system allows novel ways to combine their signals and provide enhanced localization and characterization tissues in real time.

### SUMMARY

Some embodiments of the systems and methods provided herein expand the functionality of the previously disclosed multimodality surgical probe system provided by U.S. Non-Provisional patent application Ser. No. 18/668,205 by incorporating autofluorescence detection. The enhanced probe and associated control unit permit real-time acquisition, processing, and display of autofluorescence emission data. This addition enables visualization of tissue health and pathological changes without contrast agents, facilitating identification of malignancies, margins, or tissue perfusion in surgical settings.

The autofluorescence modality is incorporated through a modular excitation and detection system located at the distal end of the probe. The excitation light source emits in a wavelength range that excites intrinsic tissue fluorophores. Emitted autofluorescence is captured by a photodetector and filtered through an optical path optimized for endogenous signal discrimination.

The control unit executes algorithms to render autofluorescence signals as either pseudo-color intensity maps or grayscale overlays on existing imaging modalities such as gamma or white light video. The display panel supports superimposed, toggled, or split-view visualization.

Some embodiments herein relate generally to surgical detection systems and more particularly to a handheld multimodality probe system capable of detecting and integrating various imaging and signal modalities (e.g., Gamma radiation, fluorescence, autofluorescence). In an example embodiment, a distal end of the probe is configured to detect broad range radiofrequency identification 64 with internal electromagnetic shielding. The system may include large universal symbols 65 to guide the surgeon in real time which end of the probe is used to detect either the radiofrequency or radiation. The system may further include an ergonomic “black box” architecture in the middle of the probe with a bi-directional remote control interface 66 to enhance intraoperative guidance and safety by the surgeon through power buttons 68 and confirming lights 69.

In some embodiments, the present invention further provides a Wireless Dual-Modality Probe System designed to solve ergonomic flaws of prior art. The system comprises a handheld probe featuring a central battery hub with a distinct “shoulder” region 66. User interface elements are disposed on this shoulder, medial to the high-grip zones, creating a “safety zone” that prevents accidental actuation. These user interface elements include but are not limited to on respective power buttons 68, and lights for battery and connection confirmation 69.

Critically, some embodiments of the probe utilize a “Central Hub” architecture (FIGS. 10A-10C) wherein the battery housing (62a-62c) serves a dual purpose: providing power and acting as an electromagnetic interference attenuating structure between a wireless antenna and a radiation sensor assembly 67. This may attenuate electromagnetic interference (EMI) that typically degrades signal quality in wireless gamma probes.

The system may further comprise a Control Console featuring a “Digital Mirror” (FIG. 11) Graphical User Interface (GUI) 70. This interface may create a bi-directional link, allowing a non-sterile assistant to toggle probe modes remotely. To minimize cognitive load, and be in accordance with most breast oncology procedures, the GUI may be vertically stratified: it may dedicate a top zone to Radar/RFID visualization 71 with specific Radar/RFID settings 72 and a bottom zone to high-contrast gamma rays count rate 73 and gamma ray specific settings 74. The GUI 70 will have a battery status indicator 75 and “black box” architecture 76 to configure settings or navigate to other menus.

Additionally, the system may enable a Streamlined Surgical Setup (FIG. 12b). The Compact Portable Control Console 83 may be sized to be placed on a mobile stand directly within the sterile field, allowing the Surgeon 77 to view real-time data without averting their gaze from the Patient 78.

The present invention may further provide highly specialized structural architectures to accommodate both robotic-assisted and modular open surgical procedures. In a first alternative embodiment, the apparatus may feature a flexible mode comprising a rigid proximal hub, a flexible articulating shaft constructed of high-tensile dielectric materials, and a distal sensing end. This may allow the probe to navigate intracorporeal geometries through standard surgical trocars. In a second alternative embodiment optimized for open surgery, the apparatus may feature a modular, break-apart architecture. The housing may be configured to mechanically decouple into two independent segments, each containing a dedicated internal power unit, to provide redundant power and rapid intraoperative modularity.

Aspects of the various embodiments may be implemented alone or in any combination. For example, one or more aspects of the “first alternative embodiment” may be combined with one or more aspects of the “second alternative embodiment”.

### BRIEF DESCRIPTION OF THE FIGURES

FIG. 1a is an image of a multi-modality probe according to an embodiment of the present disclosure.

FIG. 1b is a block diagram of a system of a multi-modality probe according to an embodiment of the present disclosure.

FIG. 1c is an image of a display provided by a system of a multi-modality probe according to an embodiment of the present disclosure.

FIG. 2 is an image of a display provided by a system of a multi-modality probe according to an embodiment of the present disclosure.

FIG. 3 is an image of a display provided by a system of a multi-modality probe according to an embodiment of the present disclosure.

FIG. 4 is an image of a display provided by a system of a multi-modality probe according to an embodiment of the present disclosure.

FIG. 5 is an image of a display provided by a system of a multi-modality probe according to an embodiment of the present disclosure.

FIG. 6 is an image of a display provided by a system of a multi-modality probe according to an embodiment of the present disclosure.

FIG. 7 is an image of a display provided by a system of a multi-modality probe according to an embodiment of the present disclosure.

FIG. 8 is an image of a display provided by a system of a multi-modality probe according to an embodiment of the present disclosure.

FIG. 9a is an image of a display provided by a system of a multi-modality probe according to an embodiment of the present disclosure.

FIG. 9b is an image of a display provided by a system of a multi-modality probe according to an embodiment of the present disclosure.

FIG. 10A is a cross-sectional schematic view of internal workings of a rigid modular embodiment, detailing a partitioned internal power unit housing (62a-62c) and decoupled sensor arrays.

FIG. 10B is an exterior perspective view of a unified surgical probe apparatus of FIG. 10A in a mechanically coupled, rigid configuration, illustrating ergonomic segregation of the user interface.

FIG. 10C is an exploded view of the surgical probe apparatus of FIG. 10B, illustrating the decoupled redundant battery architecture and the hermetically sealed mechanical decoupling interface.

FIG. 11 is a schematic view of an external Control Console GUI according to an embodiment of the present disclosure, illustrating a “Digital Mirror” bi-directional interface and split-screen visualization.

FIG. 12A is a diagram illustrating a conventional prior art surgical setup, depicting a surgeon's field of view broadened to two different control console GUIs of a conventional wired broad range RFID control console (80) and conventional gamma radiation control console (81).

FIG. 12B is a diagram illustrating a streamlined surgical setup of the present invention, showing the surgeon utilizing the probe with the compact control console placed on a mobile stand to view both RFID and TC-99m counts during each respective procedure on the novel control console GUI (82).

FIG. 13 is a schematic view of a surgical probe apparatus in a flexible mode, illustrating a rigid proximal hub, a bundled flexible articulating shaft assembly, and a plurality of distinct modality-specific sensors configured for simultaneous minimally invasive trocar insertion.

FIG. 14 is a distal end view of the flexible cords (84) and corresponding clustered sensor tips (85a-85c) nest cohesively to enable simultaneous, intact insertion without exceeding the maximum boundaries of the 12 mm working channel of a standard surgical trocar (86).

### DETAILED DESCRIPTION

The present disclosure provides a hand-held probe that combines multiple modalities in one assembly and provides utility to the surgeon and promotes efficiency in the operating room in general. Systems and methods described herein provide a surgical probe for use in an operating room environment with a multitude of modalities, all available on the single handheld device.

All or some of the modalities mentioned in the background section above become available to a user of the probe on a single physical device. Software and other functionality executing on the probe and on a control unit computer allows the user to alternate between these modalities by manipulating controls on the probe.

The surgical probe system described herein comprises:
A handheld probe body operable in a sterile field, containing modular interfaces for installation or removal of modality-specific components as mentioned in the parent invention.
An autofluorescence detection module comprising an excitation light source, optical filter set, and a sensitive photodetector or camera; the reasonable range of excitation would range from 300-800 nm and with lenses able to detect emission signals within 400-850 nm.
A control unit with hardware and software configured to activate the autofluorescence module, receive spectral emission signals, process the data, and display tissue autofluorescence properties;
A user interface permitting modality selection, image format configuration, and signal intensity thresholding for improved interpretation; and
A user interface permitting multiple modalities to images and counts being displayed simultaneously for real time cross checking and improvement of patient care and an increase in ease of use for the clinical staff.

The autofluorescence modality operates independently or in tandem with the existing modalities disclosed in the parent application: Tc-99, I-125, RFID, fluorescence (contrast-agent based), ultrasound, Magseed®, and white light camera. Selection and switching between modalities is performed via touchscreen command. A processor such as a single photon avalanche diode (SPAD), already integrated in Tc-99 detecting gamma probes can quickly scatter correct between different absorption levels of both radiation and light.

The autofluorescence signal display may be adapted to reflect tumor-specific signatures by identifying intensity drop-off or spectral shift, including but not limited to breast, neck (thyroid/parathyroid), colorectal, bladder, liver, or lung applications. Autofluorescence is also used in margin detection for suspicious lesions in cancer surgeries. This may include benign adenoma or tumor margins.

Alternatively, a clinician operating the control unit computer may handle the task of alternating between modalities upon instruction of the surgeon in the operating room. The surgeon need not even set the probe down as the single device allows access to all or some of the modalities via the activated controls.

Which modality or modalities a surgeon chooses to use, the manner and order in which he/she uses them, and how the results of the surgeon's use of the modalities are displayed on the screen in the operating room may vary from one surgeon to the next, based on each surgeon's preferences and the specifics of the surgical operation. In embodiments some surgeons may never use one or more modality. Each of these factors also depends on the type of treatment being administered, based on the patient's treatment plan determined by their care team.

Further, the probe with its modalities is modular such as hardware and software for each modality may be changed out or not installed at all. It is not mandatory that the probe provided herein be outfitted to contain components for all modalities. Some surgeons may never want a particular modality and therefore components for that unwanted modality may never be installed in the probe used by those surgeons.

The handheld probe with its modalities communicates with the nearby control unit computer. The computer directs how results of examinations using the various modalities and other information are projected on a large monitor. The monitor may be in the operating room for viewing by a physician that is using the probe.

In embodiments, the control unit may be operated by a clinician separate from the surgeon but in communication with the surgeon. In embodiments, the surgeon may verbally or electronically instruct the clinician to change the modality of the probe instead of making the change him/herself.

A probe that can detect various radioisotopes, RFID, fluorescent light, take optical images, perform ultrasonography and detect Magseed® may allow surgeons to detect multiple types of lesions through only one device in the operating room. The surgeon is provided significant flexibility and convenience by not having to alternate between physical handheld devices.

The intraoperative probe provided herein may detect a combination of 99mTc for sentinel lymph node biopsy, I-125 seed localization and RFID. The probe will allow surgeons to have more choices on breast lesion localization methods without the concerns of cost for the hospital of additional devices or sterilization hazards.

Many gamma probes can detect a combination of Tc99m for sentinel lymph nodes and tumor through I-125 filled seeds. By being able to detect 99mTcm, I-125 and RFID, the number of instruments needed by the surgeon during a cancer localization and resection comprising mastectomy or lumpectomy will be reduced, saving vital space in the operating room. Hospital staff will only need to switch between modalities in software on the control unit screen instead of changing from one physical probe to another.

The probe provided herein with its modalities connects with the control unit computer that may be situated in the surgical environment or elsewhere. A clinician accessing the computer manipulates how data gathered by the probe and its modalities is displayed on a large monitor in the operating room or on other devices.

The system may, for example, permit various arrangements of data generated by the various modalities depending on preferences of a surgeon or other professional using the probe in a surgical setting.

A circuit board is integrated into the probe for the detection modalities and is programmed to alternate between the modalities based on selection by clinician and based on control unit selection. Communication between the circuit board contained within the probe and the control unit computer may be via Bluetooth. The probe has a solid-state photomultiplier detection system for radioactive tracers (99mTc and I-125). Tungsten shielding is in place that houses the detection systems for radioactive tracers to maintain a straight spatial resolution from radioactive emissions.

An RFID detection system installed in the probe connects to the circuit board in the probe. The probe communicates with the control unit that displays radiofrequency intensity and distance in millimeters. The magnetic seed detection system in the probe is housed in metallic shielding to avoid electromagnetic interference.

As noted, the probe also contains a system for ultrasound detection. The ultrasound system comprises a transducer array, a beamformer, and a digital signal processor. A white light camera lens is available to display a live image if desired.

A fluorescence detector is located in a front area of the probe without tungsten shielding. The front window fluorescence detector must be transparent to absorb light for fluorescence detection and not interfere with gamma detection.

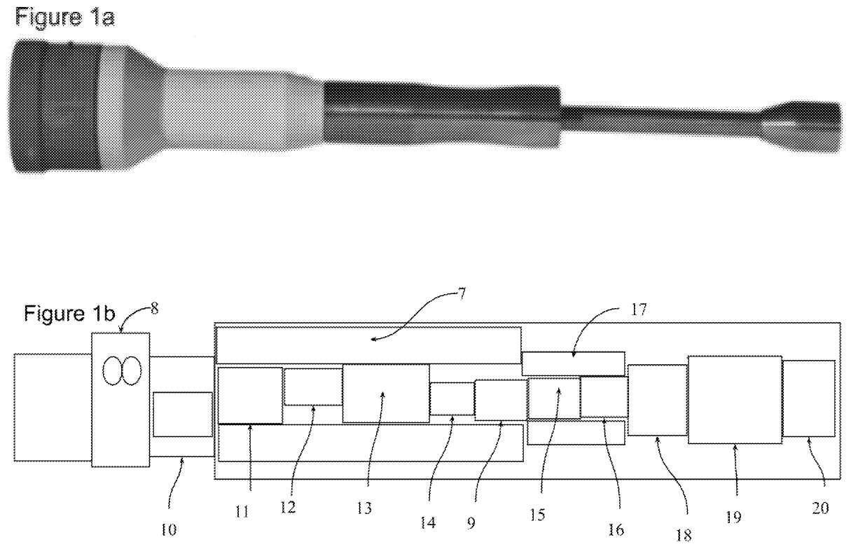

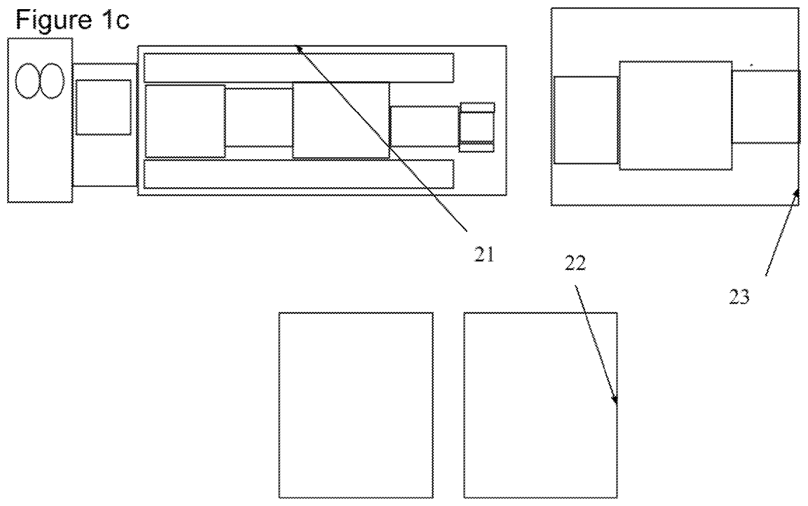

Turning to the figures, FIG. 1a and FIG. 1b illustrate objects and interactions of a system 100, specifically the probe and its objects as described above. FIG. 1a illustrates the exterior of the probe 6 with a wider portion of the probe to the left of the device being the area held by the user.

FIG. 1 b is a block diagram illustrating one or more modalities and other objects of the system provided herein. Components of the illustrated probe 6 include a battery 8, circuit boards 10, an RFID Detection system 11, a magnetic sensor detection system 12, SiPM-scintillator for radiation detection 13, a scintillator 14, a SiPM for autofluorescent light 15, autoflorescent light sources and filter apparatus (including multiple light sources and corresponding lenses to absorb fluorophore emissions) 16, shielding for autoflorescent detection apparatus such as polyethylene glycol (PEG on sides to keep unwanted light sources away) 17, fluorescence detection system 18, ultrasound detection 19, and a white light camera 20. Tungsten shielding 7 also covers one or more objects as illustrated in FIG. 1b. The acronym SSPM or SiPM both indicate solid-state photomultiplier.

FIG. 1c illustrates a locking mechanism, specifically the object 21, which is a locking mechanism on the main body of the probe that can attach and reattach systems for ultrasound, fluorescence, and white light camera, assembled into a separate section 23, to a main body of the probe. This brings utility for clinicians to have a smaller probe diameter size based on the case, since ultrasound, fluorescence, and white light detectors may increase the probe's diameter.

The type of locking mechanism associated with object 21 may conserve sterilizations. For example, there may be a recess for locking with regular/known methods. This could also be done by fitting connectors that can make contact with relevant circuitry in the probe. A cross-section of the locking mechanism is designated at 22 in FIG. 1C. Discussion herein of the locking mechanism does not mean that such a mechanism is a mandatory object of systems and methods provided herein. Such a mechanism is optional.

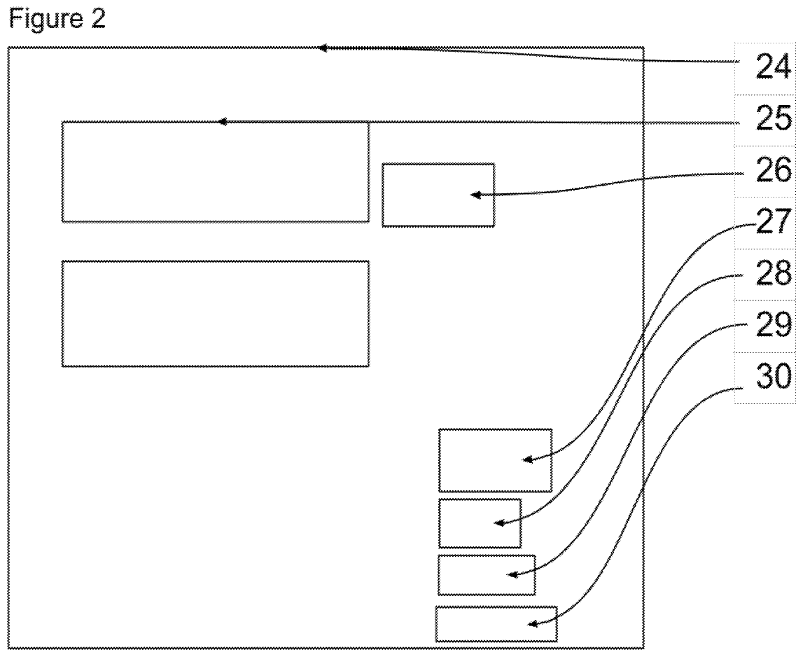

FIG. 2 is a depiction of a screen of the control unit computer described above wherein an operator may manipulate views as selected by surgeons' operating room staff. The control unit screen, indicated at 24, displays an array of count intensities from the probe depending on clinician choice. Since gamma probes are most often used during breast tumor detection and removal and sentinel node biopsies, the default intensity count displays may be 99mTc and I-125. Features that are typical for these devices include but are not limited to sonification, accumulate ten second, background count subtract, and sound zero level.

The object of FIG. 2 indicated by object 25 is an intensity count display for the I-125 SEED. Selection of this object enables a clinician to select display intensity counts of radioactive seed and distance from seed in millimeters or other measure. Objects indicated hereafter as buttons may be objects that when selected invoke functionality. Buttons may be icons, links or other executable objects.

Object 26 indicates a display for distance reading in millimenters for tumor detection. This is the distance count for either I-125 or RFID depending on the mode that is set by the clinician in the settings which is based on objects 26-30. Object 27 of FIG. 2 indicates an icon or button for clinicians to quickly simplify the screen by showing “primary counts” for the case. This could be only a set of one or two counts that are displayed.

Object 28 of FIG. 2 is an icon or button for the clinician to access a graphical menu that enables selection for immunofluorescence image display, intensity counts, or further customization of displays. For example, the clinician may enter a selection for a screen that displays all five types of intensity counts (99mTc, I-125, RFID, immunofluorescence, and autofluorescence) and live images of the green immunofluorescence intensity, autofluorescence intensities, or any other permutations thereof.

Object 29 indicates a control enabling display for count intensity. Object 30 of FIG. 2 may be a button for distance readings to switch from I-125 seed localization to RFID instantaneously. Object 26 may be a button on the control unit for clinicians to switch from “RFID” readings to I-125 seed localization for breast lesion and/or tumor detection.

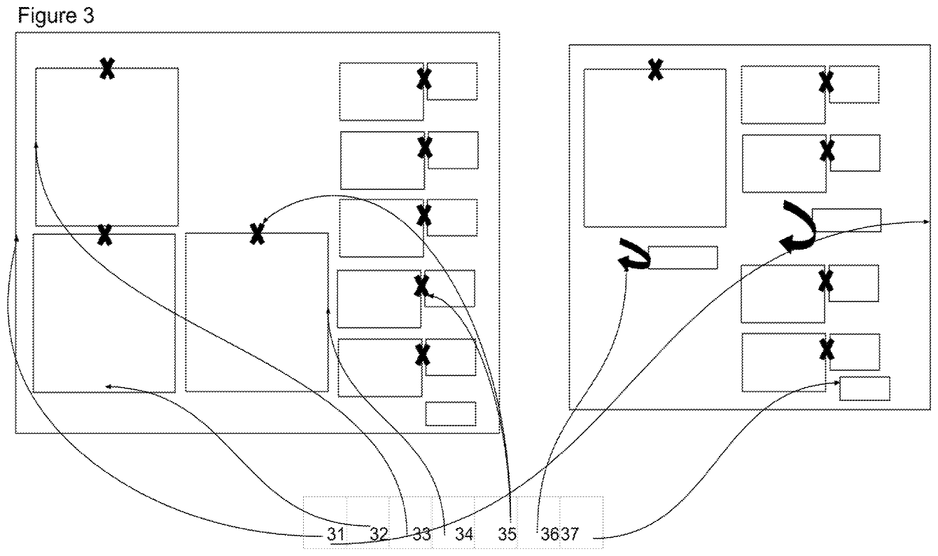

FIG. 3 is an image of screens of the control unit viewable by a clinician when assisting a surgeon using the multimodality probe. Object 31 is for the control unit screen displaying intensity count readings in small rectangles on the right. Smaller boxes on the far right are corresponding distance measurements if the modalities displayed involve that measurement (RFID and I-125). Object 32 represents green immunofluorescence intensity displayed as an image. Live images may be displayed either in the form of 99mTc heat maps, ultrasound, white light display like object 33, autofluorescence, IFG or superimposition thereof in another display like object 34.

Object 35 in FIG. 3 is a button or other selectable object for clinicians to discontinue display of intensity counts. Object 36 is a button for clinicians to redo the display they stopped. Object 37 is a button for clinicians to return to a different interface with more details of certain intensity count displays such as 99mTc which includes background count subtract and sound zero level, or the energy setup form for the radioactive tracers.

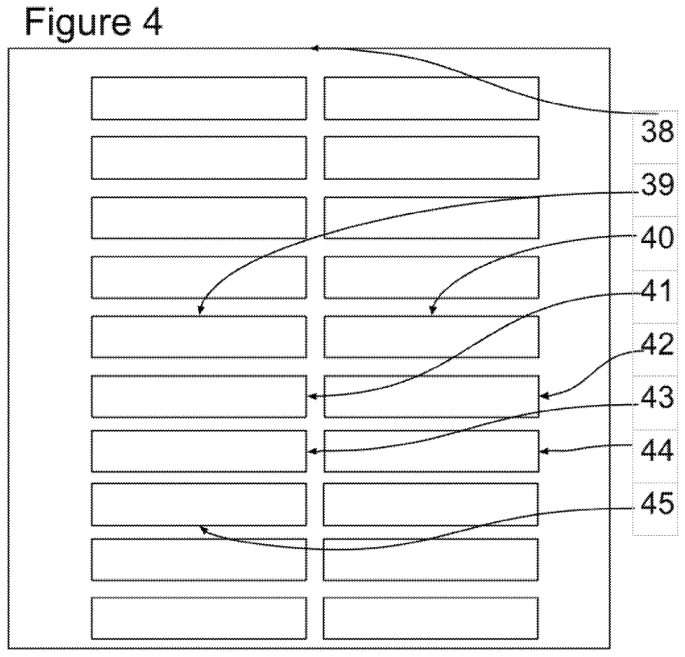

FIG. 4 is a view of a display provided in various embodiments for customizing settings for the control unit computer for switching user interface in settings in response to preference indicated by surgeons or other users. Object 38 is an image of a control unit screen illustrating different display settings. Object 39 is a button for clinicians to configure sounds for RFID signal detection and modulation.

Object 40 is a button for clinicians to configure sounds for Magseed® signal detection and modulation. Object 41 is a button for clinicians to open window for immunofluorescence green intensity display as an image. Object 42 is a button for immunofluorescence intensity display as counts. Object 43 is a button for autofluorescence intensity display as an image. Object 44 is for autofluorescence intensity display as counts. Object 45 is a button for clinicians to customize the layout of intensity displays as counts or images.

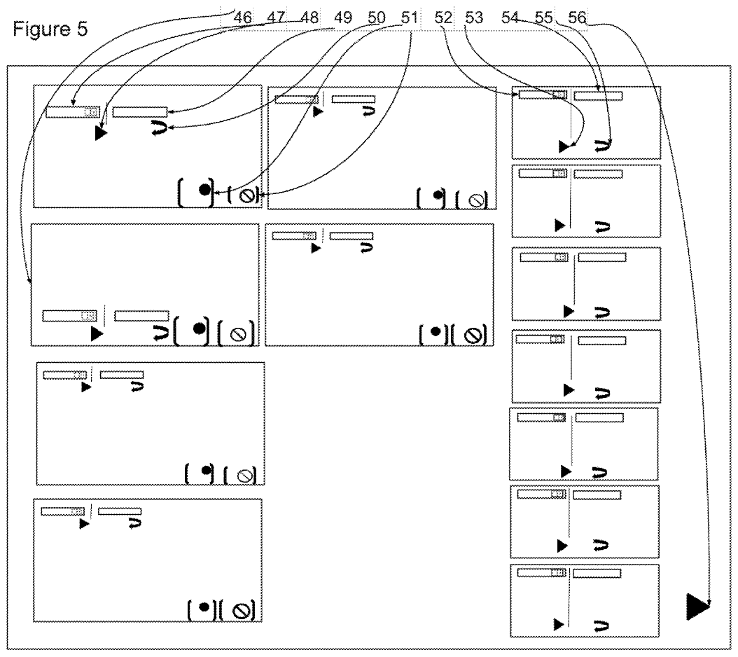

FIG. 5 is another view of displays provided by the control unit computer as it exchanges messages with the probe. Object 46 is a customization page for clinicians to select which modality is displayed on each panel from top to bottom. FIG. 5 mimics the appearance of the display as viewed by a clinician.

FIG. 5 depicts alternatives available to the surgeon. Viewing the display as shown in FIG. 5, the surgeon sees the alternatives available. In embodiments, objects appearing in the display may be superimposed atop one another. Two-dimensional object intensity images such as 99mTc, IFG and white camera may be superimposed atop one another, but not when ultrasound is involved.

Object 47 is a button for clinicians to select display image type on screen. These options are ultrasound, 99mTc intensity display, immunofluorescent green display, autofluorescent displays, white light display, or images simultaneously and superimposed to each other. For example, the surgeon can superimpose the white light visual with a 99mTc intensity display or white light visual superimposed with an immunofluorescent green display, or white light, 99mTc intensity display and immunofluorescent green display, or combinations of autofluorescent intensities. All may be superimposed at once. This may not include modalities that are mutually exclusive such as 99mTc and ultrasound or autoflorescence and white light.

Object 48 is a button to confirm the selection for that intensity image modality for that display panel in the control unit. Object 49 is an example of image writing the modality type on the other side confirming the selection of the clinician for the given display panel. Object 50 is a button for clinicians to undo selection. Text on the other side will disappear to confirm the deselection and will allow the clinician to select another modality for that display panel from the dropdown.

Object 51 is a notice to clinician that the display is auto recorded upon detection with a cancel option button for any intensity displays as images. Object 52 illustrates options for modalities that clinicians can select to display on the control unit. Clinicians can select this as a button, and it will display the options as a dropdown. The options include 99mTc, I-125, RFID, Magseed®, autoflorescence and Immunofluorescence green.

Object 53 is a button to confirm the selection for that modality for that display panel in the control unit. Object 54 is an image of writing the modality type on the other side confirming the selection of the clinician for the given display panel.

Object 55 illustrates a button for clinician to undo selection. Text on the other side will disappear to confirm the undoing of the selection and will allow the clinician to select another modality for that display panel from the dropdown. Object 56 is a button to confirm the preferences for the clinician and move to the screen with the displays in the preferred order. FIG. 5 is where clinician makes decisions for control unit display for a particular case based on treatment/procedure for patient.

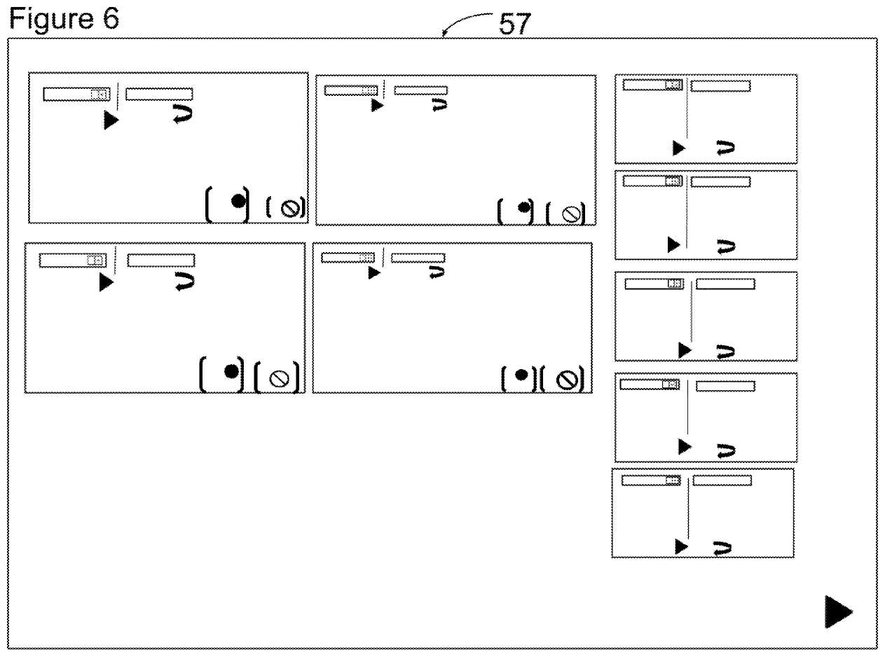

FIG. 6, also indicated by object 57, is an image of a display illustrating an embodiment of a clinician making the following settings in customization page: 99mTc intensity display, Immunofluorescent Green, and white camera superimposed on the top left, the ultrasound image displayed on the top right and the white camera displayed on the bottom left. Count screens for 99mTc and RFID are displayed.

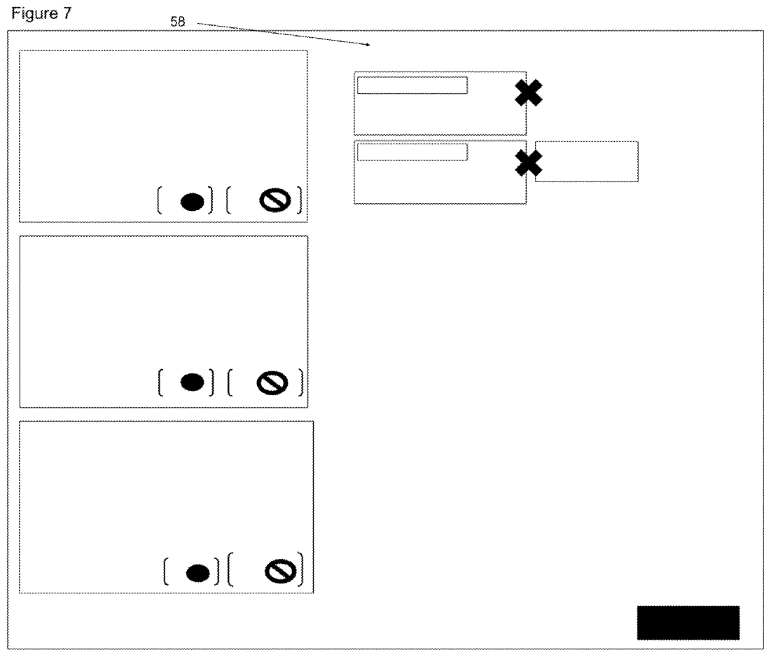

FIG. 7, also indicated by object 58, is an image of a display illustrating an embodiment of customized displays from previous examples, now in operational mode in FIG. 7.

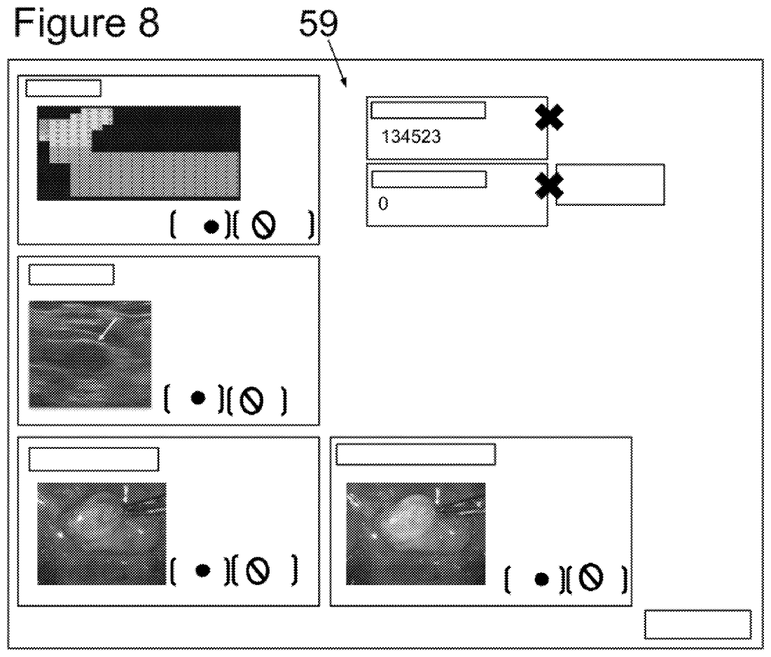

FIG. 8, also indicated by object 59, is an image of a display illustrating an embodiment of a clinician making settings in customization to utilize 99mTcm intensity display, ultrasound, immunofluorescent green intensity and white camera as well as counts for RFID and 99mTcm. The screen on the right is an embodiment of the probe being in use and being near a sentinel lymph node. Since the intensity displays that were selected can help characterize/localize sentinel nodes an image is displayed.

In FIG. 8, the 99mTcm count is high because the probe is next to a sentinel lymph node where 99mTcm has a large amount of uptake. Since the probe is not near the cancer lesion where RFID tags are placed, there is no intensity count for RFID and the distance box on the right has “-” written since the probe is not within the radius of localization of the cancer lesion.

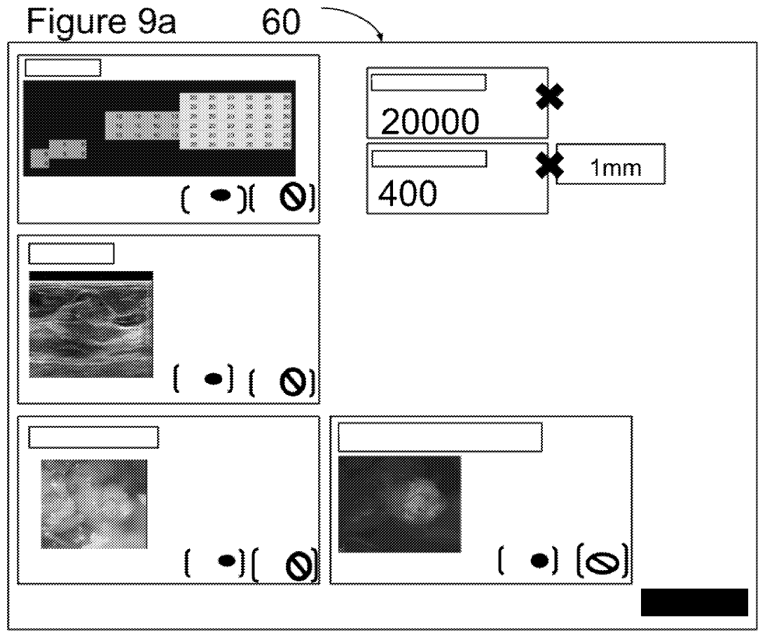

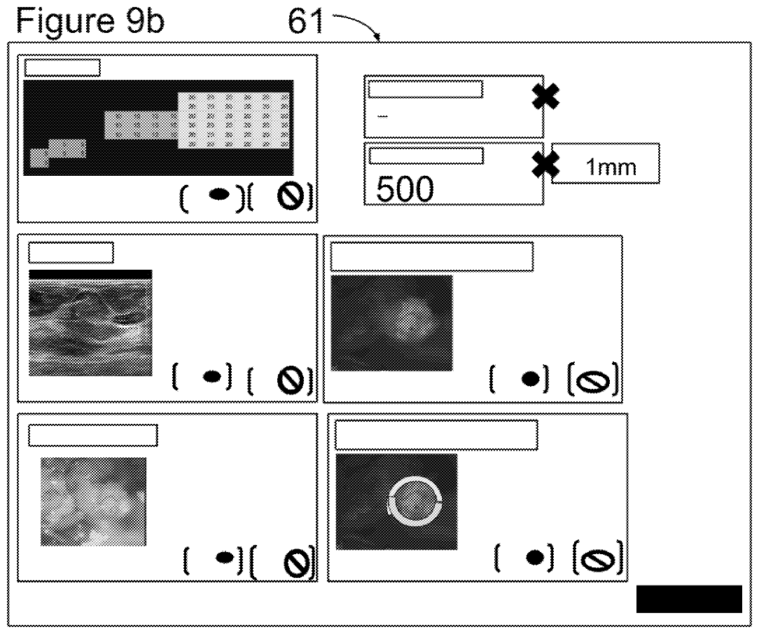

FIG. 9a, also indicated by object 60, is an image of a display illustrating an embodiment of a clinician now on a cancer tumor that has an RFID chip located and visible through the ultrasound display. A tumor is also visible in white light camera, RFID and 99mTcm intensity since the patient had the tumor injected with 99mTcm. Counts for 99mTcm are also displayed. RFID counts are also on display as well as distance measurement from the RFID chip that guides surgeons to excise the tumor.

In an embodiment, a detection multimodality probe system for use in an operating room environment is provided. The system comprises a handheld multimodality probe for at least lesion detection providing Tc99 and I-125 modalities that receives installation of an RFID detection embedded in a solid-state photomultiplier and scintillator unit of the probe. The system also receives installation of a fluorescence detection component at an end area of the probe. The system also receives installation of ultrasound, Magseed®, and white light camera functionality. FIG. 9b, also indicated by object 61, is an image of is an image of a display illustrating an embodiment of a clinician with settings that display autofluorescence Object 61's autofluorescence image uses varied colors to represent different fluorophores and their emission wavelengths. This color-coding can highlight pathological differences in tissue margins if the clinician can view a broad autofluorescence spectrum.

The probe providing access to, e.g., two or more of six modalities comprising Tc99, I.125 modality, RFID, fluorescence, ultrasound and Magseed® enables a physician in an operating room to alternate between modalities without a need to change physical devices. The probe is connected to a control unit that displays results of the probe's operations on a screen in the operating room, a layout of the displayed results configurable according to preferences of a user of the probe.

The system provides functionality, via the control unit associated with the probe, for a user to select to display intensity counts of radiofrequency methods for breast lesion and tumor detection. The probe contains a circuit board integrated for the detection modalities and programmed to alternate between the modalities based on user selection at the control unit, communication between the circuit board and the control unit via at least Bluetooth.

The RFID detection component connects via the circuit board to the control unit that configures display of both radiofrequency intensity and distance in millimeters. The modalities are interchangeable and hardware and software associated with each modality is subject to installation or removal without affecting other modalities presently installed in the probe. The fluorescence detector component is installed in a front of the probe without tungsten shielding and wherein a front window of the probe is transparent to absorb light for fluorescence detection and to not interfere with gamma detection.

In another embodiment, a multimodality probe and control unit is provided comprising a handheld multimodality probe for use in a surgical environment. The system also comprises a control unit supporting the probe. The system also comprises a customization application executing on the unit that receives a first message from the probe in an operating room, the first message containing data describing tissue observed by the probe. The system also receives a second message indicating at least one of a switching of modalities, based at least on the received second message, changes an order of panels on a display screen in the operating room. Each panel is associated with a modality and displays at least data generated by the modality. The probe and control unit include IFG display as image, 99mTc intensity displayed as image, include white light camera, include images wherein the images are at least partially superimposed, and further include images with ultrasound displayed separately but simultaneously.

Changing of the order is based on direction of a physician using the probe. The modalities comprise 99mTc, I.125, RFID, fluorescence, ultrasound, and Magseed®. The panels display at least an array of count intensities from the probe depending on user choice.

Default intensity displays are for 99mTc and I125 modalities based on projected use during tumor detection and removal and sentinel node biopsies. The control unit provides a first object entitled “I-125 SEED” promoting selection of display intensity counts of radioactive seed and distance from seed in millimeters. The control unit provides a second object, selection of which causes display of intensity counts of radiofrequency method for at least one lesion where RFID disposable is inserted into the patient.

In yet another embodiment, a method for displaying electronic content collected by a probe device in a surgical environment is provided. The method comprises a control unit computer receiving data from a handheld multimodality probe in an operating room. The method also comprises the computer, based on receiving a first instruction, activating a display device in the operating room. The method also comprises the computer, based on receiving a second instruction, directing the device to display intensity of immunofluorescence green. The method also comprises the computer, based on receiving a third instruction, displaying the intensity as counts. The method also comprises the computer, based on receiving a fourth instruction, discontinuing displaying the intensity as counts and commencing displaying the intensity as at least one image.

The method also comprises the multimodality probe observing tissue using one of 99mTc, I.125, RFID, fluorescence, ultrasound, and Magseed® technologies installed in the probe. The method also comprises the computer enabling transition to a different interface with more details of a displayed intensity count.

A displayed intensity count is associated with 99mTc technology and comprises at least one of background count subtract and sound zero level and energy setup form for radioactive tracers. The method also comprises the computer confirming display preferences of a clinician for order of technologies and transitioning to a screen with displays in the confirmed preferred order.

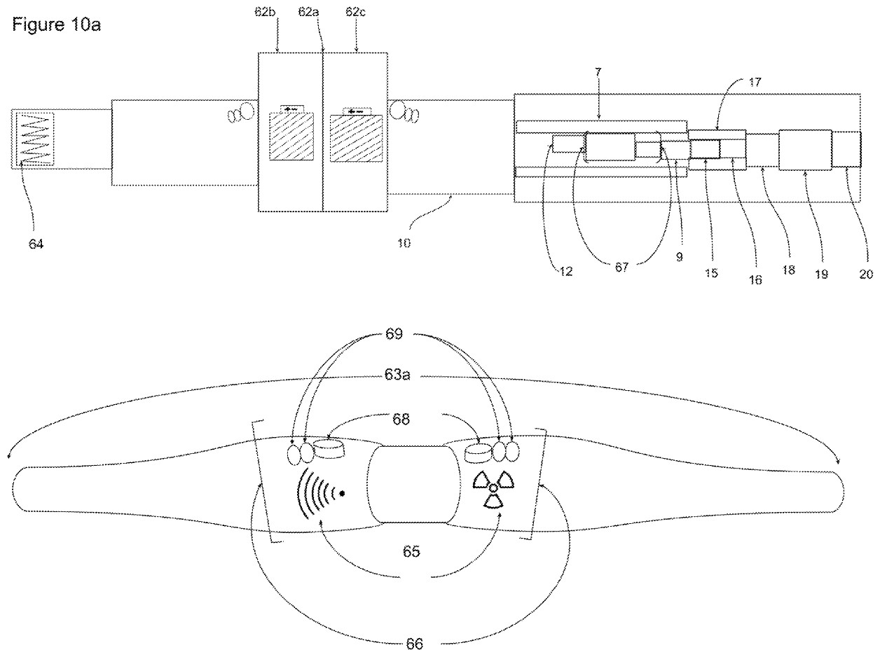

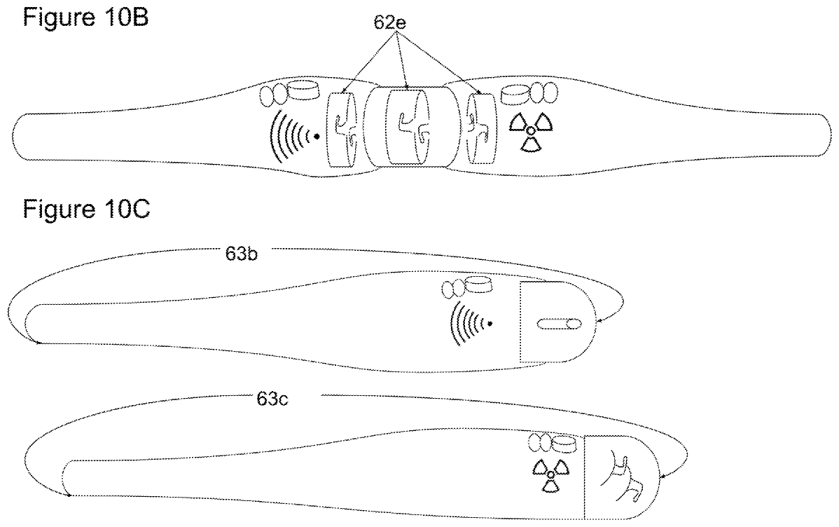

Referring to FIGS. 10A, 10B, and 10C, an embodiment of the surgical probe apparatus, or handheld probe 63, includes a modular power architecture designed for redundant operation and rapid sterilization. To facilitate the modular embodiment illustrated in FIG. 10C, a central hub 66 and a power supply 62A-62C are divided into a first internal battery 62A and a second internal battery 62B. This architecture provides independent power distribution to localized sensor arrays when the probe is physically decoupled via a secure mechanical fastening interface 62E. This fastening interface may utilize a radially engaged bayonet locking mechanism constructed from high-performance engineered thermoplastics, such as Polyether ether ketone (PEEK). To maintain a vacuum seal during oxidative sterilization protocols, including vaporized hydrogen peroxide cycles, the interface may include a redundant radial sealing architecture comprising elastomeric compression members, such as perfluoroelastomer (FFKM).

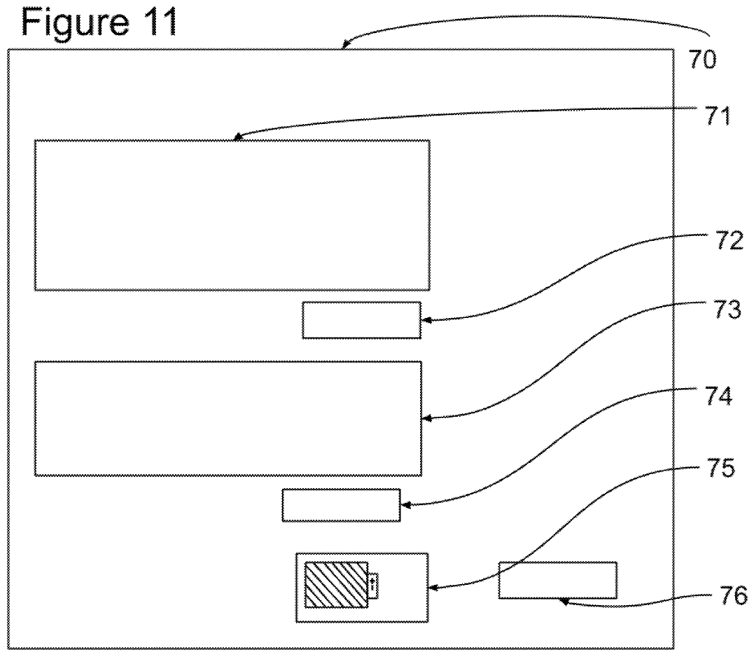

FIG. 11 illustrates an example menu with detection of the dual probe of FIGS. 10A-10C that automatically opens up when a dual ended, multimodal probe is detected. A graphical user interface (GUI) 70 detects the hardware connection and seamlessly splits the display to accommodate dual data streams. The interface includes a radar visualizer 71 and radar mode input 72 displayed simultaneously with a numeric display for radiation inputs 73 and secondary mode input 74. A system status display 75 and lateral control panel 76 organize the active inputs separate from the primary visualization zones.

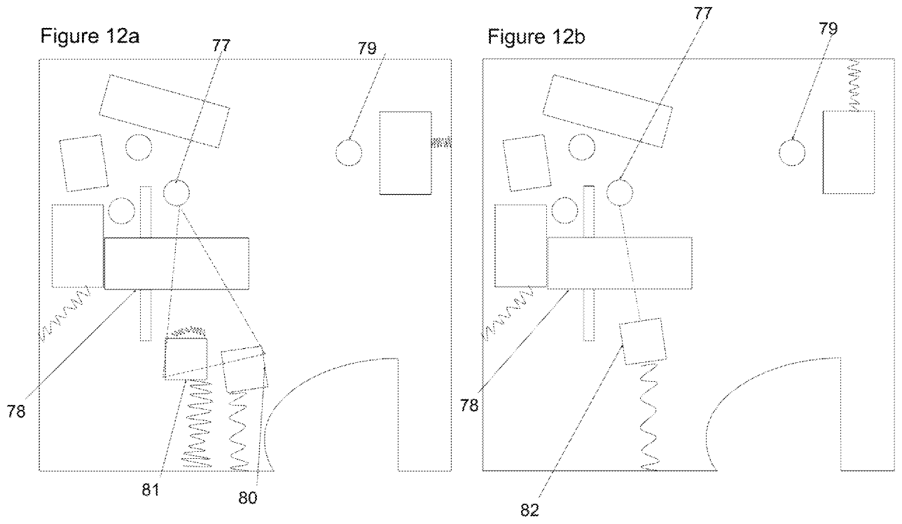

FIG. 12A illustrates a conventional operating room blueprint where a surgeon 77 operating on a patient 78 views data on two GUIs for two different probes. Each probe in the conventional operating room has its own corresponding monitor and rolling cart 81 for the wired broad range radar frequency probes. A monitor with GUI corresponding to TC-99 counts is located on a cart 80 positioned outside the immediate sterile field. The operating room circulator 79 assists the surgeon's demands that are beyond their control on the probe within the sterile field.

FIG. 12B illustrates an improved new use operating room footprint achieved by the present invention. The surgeon 77 utilizes the hand-held probe 63 on the patient 78. The system utilizes a compact control console 82 (FIG. 12B) positioned on a mobile stand. Because the dual-ended probe 63 integrates multiple modalities into a single wireless unit, the physical footprint of the equipment is drastically reduced. The OR circulating nurse 79 will only have one control console and GUI 83 to correspond with the surgeon during the case. This allows the surgeon 77 to maintain a direct line of sight to both the surgical site on the patient 78 and the diagnostic data simultaneously, mitigating visual fatigue and streamlining the surgical workflow.

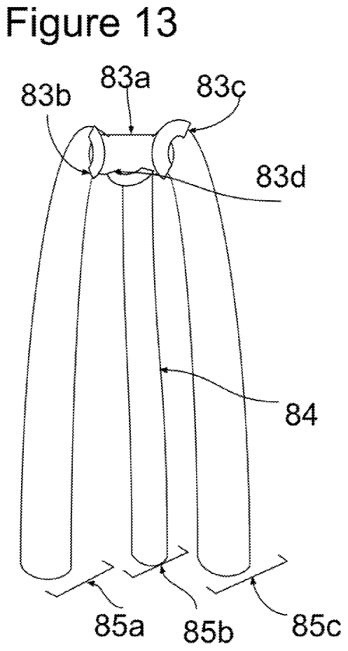

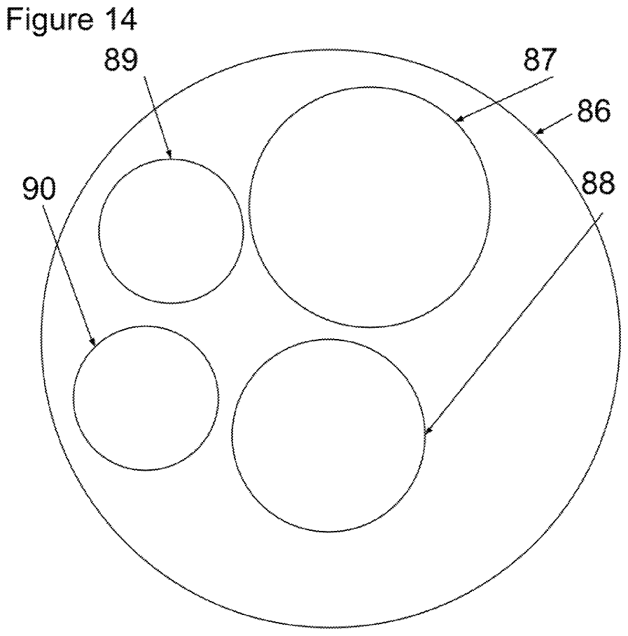

Referring to FIG. 13, an example embodiment of the handheld apparatus 63 is operable in a minimally invasive flexible mode configured to eliminate the clinical risk of detached components in the surgical field. This configuration comprises a rigid proximal hub 83a-83d, a bundled flexible articulating shaft assembly 84, and distinct, permanently affixed modality-specific sensors 85a, 85b, 85c. To optimize space within the rigid proximal hub 83a-83d, internal semiconductor arrays are integrated using high-density substrates, including engineered nanomaterial matrices or graphene-based composites. The flexible articulating shaft assembly 84 may be constructed of continuous thin polyimide films, such as Kapton, or structurally similar flexible polymer substrates. This allows the assembly to transmit power and multi-modal data while conforming to the diametrical constraints of standard surgical trocars.

Referring to FIG. 14, a transverse cross-sectional view demonstrates the dimensional viability of the flexible mode configuration. The bundled flexible cords and corresponding clustered sensor tips nest cohesively to enable simultaneous, intact insertion without exceeding the maximum boundaries of the 12 mm working channel of standard surgical trocars. FIG. 14 is a distal end view that demonstrates the dimensional viability of the flexible mode configuration. The bundled flexible cords and corresponding clustered sensor tips nest cohesively to enable simultaneous, intact insertion without exceeding the maximum boundaries of the 12 mm working channel of a standard surgical trocar (86). In this embodiment, space for a standard shaft (87) to grab and guide either a detection for gamma detection (88), broad range radiofrequency (89) or fluorescence (90).

What is claimed is:

1. A handheld surgical probe apparatus, comprising:
a housing defining a central hub portion and at least one elongated shaft portion extending therefrom;
a radiation sensor assembly disposed within the shaft portion;
a defined high grip zone located on the shaft portion; and
a user interface comprising an actuation input and a status indicator;
wherein the user interface is disposed on a shoulder surface of the central hub portion, medial to the high grip zone, such that the actuation input is spatially segregated from the high grip zone to prevent inadvertent actuation during surgical use.

2. The apparatus of claim 1, wherein the radiation sensor assembly comprises a scintillation element selected from the group consisting of Gadolinium Aluminum Gallium Garnet (GAGG), Cadmium Zinc Telluride (CZT), or Cesium Iodide (CsI).

3. The apparatus of claim 1, further comprising a Wireless Control Unit featuring a graphical user interface, wherein the interface comprises: a Radar Visualization Zone disposed in an upper region of the display; a sidebar for Radar Mode inputs; a Numeric Zone for gamma ray count rate disposed in a lower region of the display; and a Lateral Control Sidebar containing virtual actuation inputs configured to bi-directionally communicate with the probe.

4. The apparatus of claim 1, wherein the status indicator comprises a dual-LED assembly configured to provide spatially corresponding feedback indicating active sensors.

5. The apparatus of claim 1, further comprising a wireless communication module disposed within a proximal portion of the housing and a power source housing containing a battery disposed within the central hub portion; wherein the power source housing is physically interposed between the wireless communication module and the radiation sensor assembly to function as an electromagnetic interference attenuating structure, thereby attenuating electromagnetic interference generated by the wireless communication module.

6. A surgical system for intraoperative tissue detection, comprising:
the handheld probe of claim 1; and
a compact, portable control console electronically coupled to the probe, the compact, portable control console comprising an integrated display screen;
wherein the compact, portable control console is sized and configured to be positioned to provide a surgeon with a direct, unobstructed line of sight to the integrated display screen.

7. A modular multimodality surgical probe system configured for redundant power operation, comprising:
a first rigid housing segment encapsulating a first internal power unit;
at least one interchangeable sensing segment encapsulating a second internal power unit and a modality-specific sensing array; and
a mechanical fastening interface configured to selectively couple the first rigid housing segment to the at least one interchangeable sensing segment;
wherein the mechanical fastening interface comprises a radially engaged bayonet locking mechanism constructed from an oxidation-resistant engineered thermoplastic, and
wherein the interface incorporates a redundant radial sealing architecture comprising an elastomeric compression member configured to maintain a hermetic seal during vaporized hydrogen peroxide sterilization protocols.

8. The modular multimodality surgical probe system of claim 7, wherein the elastomeric compression member comprises a perfluoroelastomer (FFKM) O-ring, and wherein the at least one interchangeable sensing segment is selected from a plurality of distinct modular attachments configured for varied intraoperative spatial constraints.

9. A flexible multimodality surgical probe apparatus configured for minimally invasive procedures, comprising:
a rigid proximal hub containing an internal power unit configured as an electromagnetic attenuating structure;
a flexible articulating shaft assembly physically and electronically coupled to the rigid proximal hub, the flexible articulating shaft assembly comprising a plurality of continuous, parallel dielectric polymer substrates; and
a bundled distal sensing end comprising a plurality of permanently affixed, distinct modality-specific sensors;
wherein the flexible articulating shaft assembly and bundled distal sensing end are sized and configured for simultaneous, intact insertion through a single surgical trocar.

10. The flexible multimodality surgical probe apparatus of claim 9, wherein:
the plurality of distinct modality-specific sensors comprises at least a radiation sensor and a tissue autofluorescence sensor; and
the rigid proximal hub further comprises a selective modality activation switch configured to independently route power from the internal power unit to a single selected modality-specific sensor within the surgical trocar, thereby preventing simultaneous signal crosstalk and managing thermal output within a confined intracorporeal space.
